(19) United States
(12) Patent Application Publication
(10) Pub. No.: US 2009/0062242 A1
(43) Pub. Date: Mar. 5, 2009

(54) METHODS AND COMPOSITIONS FOR TREATING GASTROINTESTINAL CONDITIONS

(75) Inventor: John G. DEVANE, Athlone Co. Roscommon (IE)

Correspondence Address: GREENBLUM & BERNSTEIN, P.L.C., 1950 ROLAND CLARKE PLACE, RESTON, VA, 20191, US

(73) Assignee: AGI Therapeutics plc, Dublin (IE)

(21) Appl. No.: 12/199,014

(22) Filed: Aug. 27, 2008

Related U.S. Application Data
(60) Provisional application No. 60/968,377, filed on Aug. 28, 2007.

(51) Int. Cl. A61K 31/445; A61K 31/56; A61P 1/00; A61K 31/606

(52) U.S. Cl. 514/166; 514/178; 514/317

(57) ABSTRACT

Methods and compositions for treating a condition involving gastrointestinal symptoms comprising administering to a patient in need of such treatment an effective amount of a P-glycoprotein substrate, wherein the P-glycoprotein substrate is a compound exhibiting an efflux inhibition ratio (EIR) of greater than or equal to 0.4, wherein the P-glycoprotein substrate is administered in a manner to minimize bioavailability.

### CROSS-REFERENCE TO RELATED APPLICATION

This application claims priority to U.S. Provisional Application No. 60/968,377, filed on Aug. 28, 2007, the entire disclosure of which is incorporated herein by reference.

### FIELD OF THE INVENTION

The present invention relates generally to treatment of gastrointestinal conditions. The treatments involve use of compounds that are substrates for P-glycoprotein; some treatments additionally involve the additional use of non-P-glycoprotein substrates.

### BACKGROUND OF THE INVENTION

P-glycoprotein is an efflux pump that limits the permeability of certain compounds across biological barriers, such as the gastrointestinal tract and the blood-brain barrier. Compounds that are substrates for P-glycoprotein often are associated with incomplete bioavailability upon oral administration and this is particularly the case for compounds that show poor intestinal permeability characteristics. Thus, a drug like verapamil, which has high permeability and is a substrate for P-glycoprotein, is well absorbed from the length of the gastrointestinal tract and has been successfully formulated as an oral modified release dosage form with good regional gastrointestinal absorption characteristics. On the other hand, substrates for P-glycoprotein that are poorly permeable have been deemed to be poor candidates for oral administration because of limited oral absorption/bioavailability. An example of such a compound is vinblastine.

A drug like fexofenadine (an H1 antihistamine), has intermediate permeability and exhibits differential regional gastrointestinal permeability, while also being a P-glycoprotein substrate. Fexofenadine is well absorbed from the upper gastrointestinal tract due to an active transporter mechanism in the upper small intestine. Thus, for fexofenadine, permeability dominates over efflux in these absorption regions. However, at more distal gastrointestinal sites such as the lower small intestine (e.g., the ileum) and colon, where this transporter may not be as actively expressed and the expression of P-glycoprotein is higher, the efflux process dominates and absorption and oral bioavailability is reduced and incomplete. Thus, fexofenadine has been used successfully as an oral product but has not itself been formulated as a modified release formulation with release at distal gastrointestinal sites, as this would simply reduce oral bioavailability and limit therapeutic efficacy.

Thus, the formulation and presentation of P-glycoprotein substrate compounds that are poorly permeable at distal gastrointestinal sites, such as fexofenadine and other compounds, has been focused on upper gastrointestinal release rather than modified release. Thus, these compounds, if desired as modified release, are targets for gastro-retentive or upper small intestinal-retentive systems, rather than modified release formulations that transit to the distal gastrointestinal tract. The goal of these retentive systems is to enhance the absorption and systemic exposure over an extended period of release.

Alternatively, in an effort to maximize bioavailability of these P-glycoprotein substrates, efforts have been made to incorporate inhibitors of P-glycoprotein into distal controlled release forms of these compounds. Such P-glycoprotein inhibitors have the effect of increasing bioavailability. Agents that have inhibitory effects on P-glycoprotein include, for example, ketaconazole.

The use of these techniques to improve permeability, by altering the barrier nature of the gut (through penetration enhancers), or the partition characteristics of a compound into and through the enterocytes (through non-covalent complexation or covalent modification), are all directed to overcoming the poor absorption of these classes of compounds and enhancing the systemic circulation. Thus, reduced oral absorption and bioavailability has historically been viewed as a therapeutic disadvantage of traditional modified release approaches for these compounds and new inventions are directed to overcoming these perceived “deficiencies.”

It has presently been realized that these “deficiencies” can be used as advantages, and if properly formulated, the P-glycoprotein substrates can be used to achieve a desired local effect in the gastrointestinal tract, while minimizing—or entirely avoiding—systemic exposure to the drug.

### SUMMARY OF THE INVENTION

The present invention provides methods and formulations for treating gastrointestinal conditions, which reduce or entirely avoid, systemic exposure. The present invention is advantageous, at least, in that it allows for a local therapeutic effect in the gastrointestinal tract, while minimizing systemic effects of the drug.

These features and advantages are provided at least by the following specific embodiments, which are provided for purposes of illustration so that they may be expanded upon. The invention provides methods of treating at least one condition involving gastrointestinal symptoms by administering to a patient in need of such treatment an effective amount of a P-glycoprotein substrate, wherein the P-glycoprotein substrate is a compound exhibiting an efflux inhibition ratio (EIR) of greater than or equal to 0.4, wherein the P-glycoprotein substrate is administered in a manner to minimize bioavailability. The P-glycoprotein substrate may be a compound exhibiting an EIR of greater than or equal to 0.5, 0.6, 0.7, 0.8, 0.9, or higher. Preferably, the P-glycoprotein substrate is administered in a manner to result in less than or equal to about 80%, of the bioavailability of an orally administered rapid release dosage form of the P-glycoprotein substrate. More preferably, the P-glycoprotein substrate is administered in a manner to result in less than or equal to about 70%, or 60%, or 50%, or 40%, or less, of the bioavailability of an orally administered rapid release dosage form of the P-glycoprotein substrate.

The P-glycoprotein substrate may be administered in a pharmaceutical formulation that releases less than or equal to about 20% of the P-glycoprotein substrate in up to 2 hours of testing in pH 1.2 in a USP Type 2 dissolution testing apparatus. The P-glycoprotein substrate may be administered in a pharmaceutical formulation that releases greater than or equal to about 70% of the P-glycoprotein substrate in 2 hours of testing in pH 1.2 followed by 2 hours at pH 7.2, in a USP Type 2 dissolution testing apparatus.

The P-glycoprotein substrate may be chosen from histamine H1 antagonists, corticosteroids, glucocorticosteroids, aminosalicylates, mast cell stabilisers, and leukotriene antagonists. Preferably, the P-glycoprotein substrate is chosen from histamine H1 antagonists, and preferably, it is fexofenadine.

The method may further involve administering at least one non-P-glycoprotein substrate, wherein the non-P-glycoprotein substrate is a compound exhibiting an EIR of less than 0.4. Preferably, the non-P-glycoprotein substrate is chosen from histamine H1 antagonists, corticosteroids, glucocorticosteroids, aminosalicylates, mast cell stabilisers, and leukotriene antagonists, and preferably, the non-P-glycoprotein substrate is a cromone such as cromoglycate.

The invention also provides methods of treating a gastrointestinal condition by orally administering to a patient in need of such treatment a pharmaceutical formulation comprising fexofenadine, which releases from about 0 to about 20% of the fexofenadine in the formulation within two hours of administration, wherein the bioavailability of the fexofenadine in the formulation is less than or equal to about 80% of the bioavailability of an orally administered rapid release dosage form of fexofenadine. The pharmaceutical formulation may release from about 0 to about 10% of the fexofenadine in the formulation within two hours of administration. The bioavailability of the fexofenadine in the formulation is preferably less than or equal to about 70%, 60%,50%, 40%, 30%, 20%, or less, of the bioavailability of an orally administered rapid release dosage form of fexofenadine. The pharmaceutical formulation may further comprise at least one non-P-glycoprotein substrate, wherein the non-P-glycoprotein substrate is a compound exhibiting an EIR of less than 0.4. Preferable non-P-glycoprotein substrates are chosen from anti-H1 antagonists, corticosteroids, glucocorticosteroids, aminosalicylates, mast cell stabilizers, and leukotriene antagonists.

The invention also provides oral drug delivery systems consisting of a P-glycoprotein substrate exhibiting an EIR of greater than 0.4; and pharmaceutical excipients that result in release of less than or equal to about 20% of the P-glycoprotein substrate in up to 2 hours of testing in pH 1.2 in a USP Type 2 dissolution testing apparatus. Preferably, the P-glycoprotein substrate is fexofenadine.

Also provided are oral drug delivery systems consisting of: a P-glycoprotein substrate exhibiting an EIR of greater than 0.4 and a non-P-glycoprotein substrate exhibiting an EIR of less than 0.4; and pharmaceutical excipients that result in release of less than or equal to about 20% of the P-glycoprotein substrate in up to 2 hours of testing in pH 1.2 in a USP Type 2 dissolution testing apparatus. Preferably, the P-glycoprotein substrate is fexofenadine and the non-P-glycoprotein substrate is cromoglycate.

Other features and advantages of the present invention will be set forth in the description of invention that follows, and will be apparent, in part, from the description or may be learned by practice of the invention. The invention will be realized and attained by the compositions, products, and methods particularly pointed out in the written description and claims hereof.

### DETAILED DESCRIPTION OF THE INVENTION

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

Prior to beginning the more detailed description of the invention, an understanding of some terms to be used in this description may be particularly helpful. Throughout this disclosure, reference will be made to EIR, or of efflux inhibition ratio. Efflux inhibition ratio is the ratio of a compound's permeability due to P-glycoprotein-mediated efflux activity compared to its passive permeability only.

EIR is determined using procedures disclosed in Varma & Panchagnula, Journal of Pharmaceutical Sciences, Vol. 94, No. 8, August 2005, pages 1694-1704, hereinafter “Varma & Panchagnula,” the entire disclosure of which is incorporated herein by reference. For convenience, the details of the EIR determination procedure from Varma & Panchagnula are reproduced as follows.

### Animals

Sprague-Dawley rats (270-350 g) are used to perform in situ single-pass perfusion.

### Solubility Studies

Equilibrium solubility of drugs is determined by the shake-flask method (n=3). An excess amount of the drug is added in pH 7.4 10 mM phosphate buffer and equilibrated at 37±0.2° C. with vigorous shaking in a shaker water bath (such as, for example, Julabo, Germany) for 24 h. Samples are filtered using a 0.22-μm filter (such as, for example, Millipore, USA). Aliquots of each filtrate were diluted appropriately and analyzed by UV spectrophotometry (such as, for example, DU 640i Beckman, USA).

Permeability Studies: In situ Perfusion Experiments

The surgical procedure and the in situ single-pass perfusion experiments are performed according to the methods described in Fagerholm et al., Pharm Res 13:1336-1342 (1996); Hanafy et al., Eur J Pharm Sci 12:405-415 (2001); and Fagerholm et al. J Pharm Phamacol 49:687-690 (1997). Briefly, rats are fasted for 16-18 h, prior to study, with tap water ad libidum. After anesthesia via intraperitoneal administration of, for example, thiopental sodium (50 mg/kg), rats are placed on a heating pad to maintain body temperature at 37° C. The intestines of the rats are exposed by a midline abdominal incision, and a 12-15 cm ileum segment (5 cm above the ileoceceal junction) is isolated and cannulated at both ends with glass tubing. The segment is rinsed with phosphate buffered saline (10 mL) and the perfusion solution maintained at 37° C. is pumped at a flow rate of 0.1 mL/min using a syringe pump (Harvard Apparatus PHD 2000 pump, MA, USA). The perfusion solution (pH 7.4) is NaCl 48 mM, KCl 5.4 mM, Na₂HPO₄ 2.8 mM, NaH₂PO₄ 4 mM and D-glucose 1 g/L; and contains drugs with or without quinidine (200 μM) as a P-glycoprotein inhibitor. The concentrations of compounds in the perfusion solution should be low enough to avoid precipitation in the lumen during the perfusion studies. Insoluble compounds may be added as stock solutions of DMSO, giving a final solvent concentration of <0.2%. Higher concentrations are used for highly soluble high-dose compounds considering the in vivo availability of the compound in the solution at the absorption site.

A two-step perfusion procedure is followed to determine the permeability of compounds with and without the P-glycoprotein inhibitor. This includes sampling every 5 min for a 30-min perfusion period with perfusion solution containing test compound only, after 20 min equilibrium, and then switching to perfusion solution containing both the test compound and inhibitor, and similarly sampling every 5 min for a 30-min perfusion period after a 20-min equilibrium period. Permeability is determined in four individual rats (n=4) for each compound. Out of four rats (n=4) used for each compound, two rats are first perfused with only test compound solution and then switched to perfusion solution containing both the test compound and P-glycoprotein inhibitor, while the other two rats are first perfused with solution containing both the test compound and P-glycoprotein inhibitor and then switched to perfusion solution containing only compound. Equilibrium of 20 min prior to sampling should be sufficient for both washout and to reach an initial steady state. Water flux is quantified by weight and volume measurements. To further keep a check on the intra- and inter-individual variability and presence of steady state, permeability of propranolol (passive, highly permeable), frusemide (passive, low permeable), and D-glucose (carrier-mediated, highly permeable) is monitored, by co-perfusing along with each individual compounds without and with P-glycoprotein inhibitor.

### Bioanalysis by RP-HPLC

Quantifications can be performed by RP-HPLC with dual wavelength UV detector; D-glucose can be estimated using, for example, a GOD-POD-based assay kit (such as, for example, AUTOZYME, ACCURex Biomedical Pvt. Ltd., Mumbai, India). HPLC can be conducted, for example, on Waters equipment (Waters Corp., MA, USA) equipped with 600 controller pumps, Waters 2487 Uv-Vis dual λ absorbance detector, and configured to Millenium software. The perfusion sample can be loaded onto the column by means of, for example, a 717ₚₗᵤₛ autosampler (Waters Corp., MA, USA). Chromatography can be performed, for example, on a 5-μm, 4.6×250-mm SYMMETRY C₁₈ column (Waters Corp., MA, USA) attached with 5 μm, 3.9×20 mm SYMMETRY C₁₈ guard column (Waters Corp., MA, USA). The methods can be optimized and validated for estimating individual compounds or for simultaneous analysis. For chromatography of digoxin, sulindac, imipramine, propranolol, and frusemide, the mobile phase can be 25% methanol, 25% acetonitrile, and 50% pH 3.0 (20 mM) acetate buffer, pumped on an isocratic mode at a flow rate of 0.5 mL/min with UV monitoring at 220 and 275 nm. For paclitaxel, quinidine, propranolol, and frusemide, the mobile phase can be 70% methanol, 26% pH 5.0, 20 mM acetate buffer, and 4% isopropyl alcohol, pumped at a flow rate of 0.6 mL/min and chromatograms recorded at 220 and 230 nm. In the case of L-phenylalanine, hydrochlorothiazide, ranitidine, and fexofenadine, the mobile phase can be 52% methanol, 41.7% pH 5.0 (20 mM) acetate buffer, and 6.3% isopropyl alcohol, pumped at a flow rate of 0.6 mL/min and chromatograms recorded at 230 and 275 nm. Validation of the methods can be evaluated by accuracy and precision in the working range of 50-110% of the drug concentration used in perfusion solution for individual compounds.

### Data Treatment and Statistics

Permeabilities (without and with P-glycoprotein inhibitor) are calculated after correcting the outlet concentration for water flux on the basis of the ratio of volume of perfusion solution collected and infused for each sampling point (5 min).

$${P}_{eff , Control } ( or ) {P}_{eff , Inh} = [ ( \frac{{C}_{in}}{{C}_{out}} ) - 1 ] / 2 π rl \qquad ( 1 )$$

where Q is the flow rate, Cᵢₙ and Cₒᵤₜ are the respective inlet and outlet concentration, r is radius of intestine (0.21 cm), and l is length of intestine measured after completion of perfusion.

The permeability obtained by in situ perfusion without and with P-glycoprotein inhibitor can be described by equations 2 and 3, respectively.

P_{eff,Control}=P_{PD}−P_{P-gp} (2)

P_{eff,Inh}=P_{PD} (3)

Assuming one-site Michaelis-Menten saturation kinetics for P-glycoprotein substrates, EIR can be obtained by

$$E I R = \frac{{P}_{P \text{-} gp}}{{P}_{PD}} = 1 - \frac{{P}_{eff , Control}}{{P}_{eff , Inh}} \qquad ( 4 )$$

Thus, EIR quantifies the passive drug transport attenuation by P-glycoprotein across the intestinal enterocytes. EIR provide information on the extent to which P-glycoprotein influences intestinal absorption of P-glycoprotein substrates across the enterocytes. EIR=0.75 (value lies between 0 and 1) indicates that P-glycoprotein-mediated efflux transport (P_{P-gp}) attenuates the passive permeability (P_{PD}) by 75%.

Having now provided some background terminology for understanding the invention, the invention will now be described. The invention is directed to, among other things, compositions and methods for treating gastrointestinal conditions with compounds that are P-glycoprotein substrates. Preferred compounds of the invention are those compounds exhibiting an efflux inhibition ratio (EIR) of greater than or equal to 0.4. More preferably, the P-glycoprotein substrates exhibit an EIR of greater than 0.5, 0.6, 0.7, 0.8, 0.9, or higher.

As used herein, “treating” means alleviating at least one symptom associated with the disease or condition for which the drug is administered. A “therapeutically effective amount” of a P-glycoprotein substrate is the amount of P-glycoprotein substrate (including isomers thereof and pharmaceutically acceptable salts thereof, alone or together), which alone or in combination with other drugs, provides any therapeutic benefit in the prevention, treatment, and/or management of one or more gastrointestinal conditions described below, which are amenable to prevention, treatment, and/or management with a P-glycoprotein substrate.

The invention is also directed to methods, and formulations for use in such methods, for treating a gastrointestinal condition comprising orally administering to a patient in need of such treatment a pharmaceutical formulation comprising fexofenadine, which releases from about 0 to about 20% of the fexofenadine in the formulation within two hours of administration, wherein the bioavailability of the fexofenadine in the formulation is less than or equal to about 80% of the bioavailability of an orally administered rapid release dosage form of fexofenadine. The pharmaceutical formulation may further comprise at least one non-P-glycoprotein substrate, wherein the non-P-glycoprotein substrate is a compound exhibiting an EIR of less than 0.4.

The invention also provides oral drug delivery systems consisting of a P-glycoprotein substrate exhibiting an EIR of greater than 0.4; and pharmaceutical excipients that result in release of less than or equal to about 20% of the P-glycoprotein substrate in up to 2 hours of testing in pH 1.2 in a USP Type 2 dissolution testing apparatus. Also provided are oral drug delivery systems consisting of a P-glycoprotein substrate exhibiting an EIR of greater than 0.4 and a non-P-glycoprotein substrate exhibiting an EIR of less than 0.4; and pharmaceutical excipients that result in release of less than or equal to about 20% of the P-glycoprotein substrate in up to 2 hours of testing in pH 1.2 in a USP Type 2 dissolution testing apparatus.

The methods and formulations of the invention preferably minimize bioavailability of the P-glycoprotein substrate. Ideally, the bioavailability of the P-glycoprotein substrates administered according to the present invention is less than 80% of the bioavailability of an orally administered rapid release dosage form of the same drug. More preferably, the bioavailability is less than 70%, 60%, 50%, 40%, 30%, 20%, 10%, or less, than the bioavailability of an orally administered rapid release dosage form of the same drug.

P-glycoprotein substrate exhibiting an EIR greater than 0.4 include compounds chosen from histamine H1 antagonists, corticosteroids, glucocorticosteroids, aminosalicylates, mast cell stabilisers, and leukotriene antagonists. Preferred classes of compounds include histamine H1 antagonists. Specific compounds exhibiting an EIR of greater than 0.4 include, but are not limited to, fexofenadine (a histamine H1 antagonist). It should be noted that the express inclusion of classes of compounds, or specific compounds within a class, in this specification is intended as a disclosure and contemplation of the express exclusion of those same compounds or classes of compounds. For example, the express exclusion of corticosteroids and glucocorticosteroids is contemplated. This rule of express inclusion, contemplated exclusion, applies throughout this specification.

It is also noted that some compounds, such as fexofenadine, are available as a racemic mixture of the (R) and (S) stereoisomers. The present invention contemplates the use of both racemic fexofenadine (as well as other racemic drugs) and enriched (S)-fexofenadine. As used herein, the term “enriched (S)-fexofenadine” means fexofenadine compositions in which the (S) stereoisomer is present in greater amounts than the (R) stereoisomer. For example, enriched (S)-fexofenadine comprises 51% or greater (S)-fexofenadine, such as about 55%, 60%, 65%, 70%, 75%, 80%, 85%, 90%, 91%, 92%, 93%, 94%, 95%, 96%, 97%, 98%, and 99% or greater percent of (S)-fexofenadine. The term “enriched (S)-fexofenadine” encompasses “substantially pure (S)-fexofenadine.” “Substantially pure (S)-fexofenadine,” as used herein, means a preparation of fexofenadine containing at least 90% (S)-fexofenadine.

Non-P-glycoprotein substrates exhibiting an EIR less than 0.4 also include compounds chosen from histamine H1 antagonists, corticosteroids, glucocorticosteroids, aminosalicylates, mast cell stabilizers, and leukotriene antagonists. Preferred classes of compounds include mast cell stabilizers. Specific compounds exhibiting an EIR of less than 0.4 include, but are not limited to, cromones such as cromoglycate, 4-aminosalicylic acid (4-ASA), and 5-aminosalicylic acid (5-ASA).

A formulation may include pharmaceutically active ingredients consisting of one P-glycoprotein substrate and one non-P-glycoprotein substrate. A preferred combination consists of a P-glycoprotein substrate and a non-P-glycoprotein substrate. A particularly preferred combination is a histamine H1 antagonist and a mast cell stabilizer, such as fexofenadine (histamine H1 antagonist) and cromoglycate (mast cell stabilizer).

This invention relates to formulations and methods for treating gastrointestinal conditions. Gastrointestinal conditions treatable with the present invention include, but are not limited to, food allergies, celiac disease, irritable bowel disease, mastocytosis, atopic dermatitis, inflammatory bowel disease, ulcerative colitis, granulomatous enteritis, Crohn's disease, infectious diseases of the small and large intestine, pyloric spasm, abdominal cramps, functional gastrointestinal disorders, mild dysenteries, diverticulitis, acute enterocolitis, neurogenic bowel disorders, including the splenic flexure syndrome and neurogenic colon, spastic colitis, cysts, polyps, and carcinoma, conditions with increased or altered gut secretory function, which is normally associated with cancer-related diarrhea (e.g., colon cancer), carcinoid syndrome, chemotherapy and radiotherapy linked diarrhea, acquired immune deficiency syndrome (AIDS) related diarrhea, infectious diarrhea (such as bacterial and viral), food intolerance and malabsorption related diarrhea, medicine linked diarrhea including antibiotics, celiac disease, and endocrine diseases such as Addisons disease related diarrhea or conditions of an abnormal increased mixed secretory/motility basis such as irritable bowel syndrome (IBS) and further for example, diarrhea-related or linked symptoms, chronic diarrhea, functional diarrhea, diarrhea related symptoms of inflammatory bowel disease and microscopic colitis, and/or symptoms of any of the foregoing.

The methods of the present invention may include the use of modified-release formulations. As used herein, the term “modified-release” formulation or dosage form includes pharmaceutical preparations that achieve a desired release of the drug (P-glycoprotein or non-P-glycoprotein substrate) from the formulation. A modified-release formulation can be designed to modify the manner in which the active ingredient is exposed to the desired target. For example, a modified-release formulation can be designed to focus the delivery of the active agent entirely in the distal large intestine, beginning at the cecum, and continuing through the ascending, transverse, and descending colon, and ending in the sigmoid colon. Alternatively, for example, a modified-release composition can be designed to focus the delivery of the P-glycoprotein or non-P-glycoprotein substrate in the proximal small intestine, beginning at the duodenum and ending at the ileum. In still other examples, the modified-release formulations can be designed to begin releasing active agent in the jejunum and end their release in the transverse colon. The possibilities and combinations are numerous, and are clearly not limited to these examples.

The term “modified-release” encompasses “extended-release” and “delayed-release” formulations, as well as formulations having both extended-release and delayed-release characteristics. An “extended-release” formulation can extend the period over which P-glycoprotein or non-P-glycoprotein substrate is released or targeted to the desired site. A “delayed-release” formulation can be designed to delay the release of the pharmaceutically active compound for a specified period. Such formulations are referred to herein as “delayed-release” or “delayed-onset” formulations or dosage forms. Modified-release formulations of the present invention include those that exhibit both a delayed- and extended-release, e.g., formulations that only begin releasing after a fixed period of time or after a physicochemical change has occurred, for example, then continue releasing over an extended period.

As used herein, the terms “rapid-release formulation” and “immediate-release formulation” are meant to describe those formulations in which more than about 70% of active ingredient is released from the dosage form in less than about 1 hour. Such formulations may also be referred to herein as “conventional formulations.”

The formulations of the present invention are intended to include formulations that are generic to treating all types of gastrointestinal conditions, and thus target their contents to both the small intestine and the large intestine. Other formulations within the scope of the invention include those that are more specifically designed for treating a specific disease. For example, a formulation for treating ulcerative colitis can be designed to deliver its contents entirely to the colon.

The formulations of the present invention can exist as multi-unit or single-unit formulations. The term “multi-unit” as used herein means a plurality of discrete or aggregated particles, beads, pellets, granules, tablets, or mixtures thereof, for example, without regard to their size, shape, or morphology. Single-unit formulations include, for example, tablets, caplets, and pills.

The methods and formulations of the present invention may encompass all possible combinations of components that exhibit modified- and immediate-release properties. For example, a formulation and/or method of the invention can contain components that exhibit extended-release and immediate-release properties, or both delayed-release and immediate-release properties, or both extended-release and delayed-release properties, or a combination of all three properties. For example, a multiparticulate formulation including both immediate-release and extended-release components can be combined in a capsule, which is then coated with an enteric coat to provide a delayed-release effect. Or, for example, a delayed- and extended-release caplet may comprise a plurality of discrete extended-release particles held together with a binder in the caplet, which is coated with an enteric coating to create a delay in dissolution.

The modifications in the rates of release, such as to create a delay or extension in release, can be achieved in any number of ways. Mechanisms can be dependent or independent of local pH in the intestine, and can also rely on local enzymatic activity to achieve the desired effect. Examples of modified-release formulations are known in the art and are described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566. For their disclosure relevant to modified-release formulations, the entire disclosure of these patents is incorporated herein by reference.

A number of modified dosage forms suitable for use are described below. A more detailed discussion of such forms can also be found in, for example The Handbook of Pharmaceutical Controlled Release Technology, D. L. Wise (ed.), Marcel Decker, Inc., New York (2000); and also in Treatise on Controlled Drug Delivery: Fundamentals, Optimization, and Applications, A. Kydonieus (ed.), Marcel Decker, Inc., New York, (1992), the relevant contents of each of which are hereby incorporated by reference for this purpose. Examples of modified-release formulations include but are not limited to, membrane-modified, matrix, osmotic, and ion-exchange systems. All of these can be in the form of single-unit or multi-unit dosage forms, as alluded to above.

With membrane-modified extended-release dosage forms, a semi-permeable membrane can surround the formulation containing the active substance of interest. Semi-permeable membranes include those that are permeable to a greater or lesser extent to both water and solute. This membrane can include water-insoluble and/or water-soluble polymers, and can exhibit pH-dependent and/or pH-independent solubility characteristics. Polymers of these types are described in detail below. Generally, the characteristics of the polymeric membrane, which may be determined by, e.g., the composition of the membrane, will determine the nature of release from the dosage form.

### Matrix-Based Dosage Forms

Matrix-type systems comprise an active substance of interest, mixed with either water-soluble, e.g., hydrophilic polymers, or water-insoluble, e.g., hydrophobic polymers. Generally, the properties of the polymer used in a modified-release dosage form will affect the mechanism of release. For example, the release of the active ingredient from a dosage form containing a hydrophilic polymer can proceed via both surface diffusion and/or erosion. Mechanisms of release from pharmaceutical systems are well known to those skilled in the art. Matrix-type systems can also be monolithic or multiunit, and can be coated with water-soluble and/or water-insoluble polymeric membranes, examples of which are described above.

Matrix formulations of the present invention can be prepared by using, for example, direct compression or wet granulation. A functional coating, as noted above, can then be applied in accordance with the invention. Additionally, a barrier or sealant coat can be applied over a matrix tablet core prior to application of a functional coating. The barrier or sealant coat can serve the purpose of separating an active ingredient from a functional coating, which can interact with the active ingredient, or it can prevent moisture from contacting the active ingredient. Details of barriers and sealants are provided below.

In a matrix-based dosage form in accordance with the present invention, the P-glycoprotein and/or non-P-glycoprotein substrate and optional pharmaceutically acceptable excipient(s) are dispersed within a polymeric matrix, which typically comprises one or more water-soluble polymers and/or one or more water-insoluble polymers. The P-glycoprotein and/or non-P-glycoprotein substrate can be released from the dosage form by diffusion and/or erosion. Wise and Kydonieus describe such matrix systems in detail.

Suitable water-soluble polymers include, but are not limited to, polyvinyl alcohol, polyvinylpyrrolidone, methylcellulose, hydroxypropylcellulose, hydroxypropylmethylcellulose, or polyethylene glycol, and/or mixtures thereof.

Suitable water-insoluble polymers also include, but are not limited to, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride) or polyurethane, and/or mixtures thereof.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethyl-cellulose, alginates, gelatin, polyvinyl pyrrolidone, sucrose, and acacia; humectants, such as glycerol; disintegrating agents, such as agar, calcium carbonate, potato and tapioca starch, alginic acid, certain silicates, EXPLOTAB, crospovidone, and sodium carbonate; solution retarding agents, such as paraffin; absorption accelerators, such as quaternary ammonium compounds; wetting agents, such as cetyl alcohol and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients can have more than one role or function, or can be classified in more than one group; the classifications are descriptive only, and are not intended to limit any use of a particular excipient.

In one example, a matrix-based dosage form can comprise the P-glycoprotein and/or non-P-glycoprotein substrate, a filler, such as starch, lactose, or microcrystalline cellulose (such as AVICEL™); a binder/controlled-release polymer, such as hydroxypropyl methylcellulose or polyvinyl pyrrolidone; a disintegrant, such as EXPLOTAB™, crospovidone, or starch; a lubricant, such as magnesium stearate or stearic acid; a surfactant, such as sodium lauryl sulfate or polysorbates; and a glidant, such as colloidal silicon dioxide (AEROSIL™) or talc.

The amounts and types of polymers, and the ratio of water-soluble polymers to water-insoluble polymers in the inventive formulations are generally selected to achieve a desired release profile of the P-glycoprotein and/or non-P-glycoprotein substrate, as described below. For example, by increasing the amount of water insoluble-polymer relative to the amount of water soluble-polymer, the release of the P-glycoprotein and/or non-P-glycoprotein substrate can be delayed or slowed. This is due, in part, to an increased impermeability of the polymeric matrix, and, in some cases, to a decreased rate of erosion during transit through the gastrointestinal tract.

Of course, matrix-based dosage forms may be coated with a diffusion-control membrane, such as a semi-permeable or selectively permeable membrane. Indeed, many of the formulation components described herein can be used in combination: instant release cores with diffusion-controlled membranes or matrix cores with diffusion-controlled membranes, for example.

### Osmotic Pump Dosage Forms

In another embodiment, the modified-release formulations of the present invention are provided as osmotic pump dosage forms. In an osmotic pump dosage form, a core containing P-glycoprotein and/or non-P-glycoprotein substrate and optionally one or more osmotic excipients is typically encased by a selectively permeable membrane having at least one orifice. The selectively permeable membrane is generally permeable to water, but impermeable to the P-glycoprotein and/or non-P-glycoprotein substrate. When the system is exposed to body fluids, water penetrates through the selectively permeable membrane into the core containing the P-glycoprotein and/or non-P-glycoprotein substrate and optional osmotic excipients. The osmotic pressure increases within the dosage form. Consequently, the P-glycoprotein and/or non-P-glycoprotein substrate is released through the orifice(s) in an attempt to equalize the osmotic pressure across the selectively permeable membrane.

In more complex pumps, the dosage form can contain two internal compartments in the core. The first compartment contains the P-glycoprotein and/or non-P-glycoprotein substrate and the second compartment can contain a polymer, which swells on contact with aqueous fluid. After ingestion, this polymer swells into the drug-containing compartment, diminishing the volume occupied by the P-glycoprotein and/or non-P-glycoprotein substrate, thereby forcing the P-glycoprotein and/or non-P-glycoprotein substrate from the device at a controlled rate over an extended period of time. Such dosage forms are often used when a zero order release profile is desired.

Osmotic pumps are well known in the art. For example, U.S. Pat. Nos. 4,088,864, 4,200,098, and 5,573,776, each of which is hereby incorporated by reference for this purpose, describe osmotic pumps and methods of their manufacture. Osmotic pumps of the present invention can be formed by compressing a tablet of an osmotically active P-glycoprotein and/or non-P-glycoprotein substrate, or an osmotically inactive P-glycoprotein and/or non-P-glycoprotein substrate in combination with an osmotically active agent, and then coating the tablet with a selectively permeable membrane which is permeable to an exterior aqueous-based fluid but impermeable to the P-glycoprotein and/or non-P-glycoprotein substrate and/or osmotic agent.

One or more delivery orifices can be drilled through the selectively permeable membrane wall. Alternatively, one or more orifices in the wall can be formed by incorporating leachable pore-forming materials in the wall. In operation, the exterior aqueous-based fluid is imbibed through the selectively permeable membrane wall and contacts the P-glycoprotein and/or non-P-glycoprotein substrate to form a solution or suspension of the P-glycoprotein and/or non-P-glycoprotein substrate. The P-glycoprotein and/or non-P-glycoprotein substrate solution or suspension is then pumped out through the orifice, as fresh fluid is imbibed through the selectively permeable membrane.

Typical materials for the selectively permeable membrane include selectively permeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose, dipalmitate, cellulose dioctanoate, cellulose dicaprylate, cellulose dipentanate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), cellulose acetate, cellulose diacetate, cellulose triacetate, and/or mixtures thereof.

The osmotic agents that can be used in the pump are typically soluble in the fluid that enters the device following administration, resulting in an osmotic pressure gradient across the selectively permeable wall against the exterior fluid. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium sulfate, D-mannitol, urea, sorbitol, inositol, raffinose, sucrose, glucose, hydrophilic polymers such as cellulose polymers, and/or mixtures thereof.

As discussed above, the osmotic pump dosage form can contain a second compartment containing a swellable polymer. Suitable swellable polymers typically interact with water and/or aqueous biological fluids, which causes them to swell or expand to an equilibrium state. Acceptable polymers exhibit the ability to swell in water and/or aqueous biological fluids, retaining a significant portion of such imbibed fluids within their polymeric structure, so as to increase the hydrostatic pressure within the dosage form. The polymers can swell or expand to a very high degree, usually exhibiting a 2- to 50-fold volume increase. The polymers can be non-cross-linked or cross-linked. In one embodiment, the swellable polymers are hydrophilic polymers.

Suitable polymers include, but are not limited to, poly(hydroxy alkyl methacrylate) having a molecular weight of from 30,000 to 5,000,000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde, and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; and/or mixtures of any of the foregoing.

The term “orifice” as used herein comprises means and methods suitable for releasing the P-glycoprotein and/or non-P-glycoprotein substrate from the dosage form. The expression includes one or more apertures or orifices that have been bored through the selectively permeable membrane by mechanical procedures. Alternatively, an orifice can be formed by incorporating an erodible element, such as a gelatin plug, in the selectively permeable membrane. In such cases, the pores of the selectively permeable membrane form a “passageway” for the passage of the P-glycoprotein and/or non-P-glycoprotein substrate. Such “passageway” formulations are described, for example, in U.S. Pat. Nos. 3,845,770 and 3,916,899, the relevant disclosures of which are incorporated herein by reference for this purpose.

The osmotic pumps useful in accordance with this invention can be manufactured by known techniques. For example, the P-glycoprotein and/or non-P-glycoprotein substrate and other ingredients can be milled together and pressed into a solid having the desired dimensions (e.g., corresponding to the first compartment). The swellable polymer is then formed, placed in contact with the P-glycoprotein and/or non-P-glycoprotein substrate, and both are surrounded with the selectively permeable agent. If desired, the drug component and polymer component can be pressed together before applying the selectively permeable membrane. The selectively permeable membrane can be applied by any suitable method, for example, by molding, spraying, or dipping.

### Membrane-Modified Dosage Forms

The modified-release formulations of the present invention can also be provided as membrane modified formulations. Membrane-modified formulations of the present invention can be made by preparing a rapid release core, which can be a monolithic (e.g., tablet) or multi-unit (e.g., pellet) type, and coating the core with a membrane. The membrane-modified core can then be further coated with a functional coating. In between the membrane-modified core and functional coating, a barrier or sealant can be applied. Details of membrane-modified dosage forms are provided below.

For example, the P-glycoprotein and/or non-P-glycoprotein substrate can be provided in a multiparticulate membrane-modified formulation. The P-glycoprotein and/or non-P-glycoprotein substrate can be formed into an active core by applying the compound to a nonpareil seed having an average diameter in the range of about 0.4 to about 1.1 mm, or about 0.85 to about 1 mm. The P-glycoprotein and/or non-P-glycoprotein substrate can be applied with or without additional excipients onto the inert cores, and can be sprayed from solution or suspension using a fluidized bed coater (e.g., Wurster coating) or pan coating system. Alternatively, the P-glycoprotein and/or non-P-glycoprotein substrate can be applied as a powder onto the inert cores using a binder to bind the P-glycoprotein and/or non-P-glycoprotein substrate onto the cores. Active cores can also be formed by extrusion of the core with suitable plasticizers (described below) and any other processing aids as necessary.

The modified-release formulations of the present invention comprise at least one polymeric material, which can be applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose, or polyethylene glycol, and/or mixtures thereof.

Suitable water-insoluble polymers include, but are not limited to, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride), or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers which are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment comprising a membrane-modified dosage form, the polymeric material comprises methacrylic acid co-polymers, ammonio methacrylate co-polymers, or a mixture thereof. Methacrylic acid co-polymers such as EUDRAGIT™ S and EUDRAGIT™ L (Rohm Pharma) are particularly suitable for use in the modified-release formulations of the present invention. These polymers are gastroresistant and enterosoluble polymers. Their polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material can exhibit a solubility at a pH between the pHs at which EUDRAGIT™ L and EUDRAGIT™ S are separately soluble.

The membrane coating can comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water-insoluble polymers. Alternatively, the membrane coating can comprise a polymeric material comprising a major proportion (i.e., greater than 50% of the total polymeric content) of one or more pharmaceutically acceptable water-insoluble polymers, and optionally a minor proportion (i.e., less than 50% of the total polymeric content) of one or more pharmaceutically acceptable water-soluble polymers.

Ammonio methacrylate co-polymers such as Eudragit RS and Eudragit RL (Rohm Pharma) are suitable for use in the modified-release formulations of the present invention. These polymers are insoluble in pure water, dilute acids, buffer solutions, or digestive fluids over the entire physiological pH range. The polymers swell in water and digestive fluids independently of pH. In the swollen state they are then permeable to water and dissolved actives. The permeability of the polymers depends on the ratio of ethylacrylate (EA), methyl methacrylate (MMA), and trimethylammonioethyl methacrylate chloride (TAMCI) groups in the polymer. Those polymers having EA:MMA:TAMCI ratios of 1:2:0.2 (Eudragit RL) are more permeable than those with ratios of 1:2:0.1 (Eudragit RS). Polymers of Eudragit RL are insoluble polymers of high permeability. Polymers of Eudragit RS are insoluble films of low permeability.

The ammonio methacrylate co-polymers can be combined in any desired ratio. For example, a ratio of Eudragit RS:Eudragit RL (90:10) can be used. The ratios can furthermore be adjusted to provide a delay in release of the P-glycoprotein and/or non-P-glycoprotein substrate. For example, the ratio of EUDRAGIT RS:EUDRAGIT RL can be about 100:0 to about 80:20, about 100:0 to about 90:10, or any ratio in between. In such formulations, the less permeable polymer EUDRAGIT RS would generally comprise the majority of the polymeric material.

The ammonio methacrylate co-polymers can be combined with the methacrylic acid co-polymers within the polymeric material in order to achieve the desired delay in release of the P-glycoprotein and/or non-P-glycoprotein substrate. Ratios of ammonio methacrylate co-polymer (e.g., EUDRAGIT™ RS) to methacrylic acid co-polymer in the range of about 99:1 to about 20:80 can be used. The two types of polymers can also be combined into the same polymeric material, or provided as separate coats that are applied to the core.

In addition to the EUDRAGIT™ polymers described above, a number of other such copolymers can be used to control drug release. These include methacrylate ester co-polymers (e.g., EUDRAGIT™ NE 30D). Further information on the EUDRAGIT™ polymers can be found in “Chemistry and Application Properties of Polymethacrylate Coating Systems,” in Aqueous Polymeric Coatings for Pharmaceutical Dosage Forms (ed. James McGinity, Marcel Dekker Inc., New York, pg 109-114).

The coating membrane can further comprise one or more soluble excipients so as to increase the permeability of the polymeric material. Suitably, the soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose and sucrose, sugar alcohols such as lactitol, maltitol, mannitol, sorbitol and xylitol, xanthan gum, dextrins, and maltodextrins. In some embodiments, polyvinyl pyrrolidone, mannitol, and/or polyethylene glycol can be used as soluble excipients. The soluble excipient(s) can be used in an amount of from about 0.5% to about 80% by weight, based on the total dry weight of the polymer.

In another embodiment, the polymeric material comprises one or more water-insoluble polymers, which are also insoluble in gastrointestinal fluids, and one or more water-soluble pore-forming compounds. For example, the water-insoluble polymer can comprise a terpolymer of polyvinylchloride, polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compounds can be uniformly or randomly distributed throughout the water-insoluble polymer. Typically, the pore-forming compounds comprise about 1 part to about 35 parts for each about 1 to about 10 parts of the water-insoluble polymers.

When such dosage forms come in to contact with the dissolution media (e.g., intestinal fluids), the pore-forming compounds within the polymeric material dissolve to produce a porous structure through which the P-glycoprotein and/or non-P-glycoprotein substrate diffuses. Such formulations are described in more detail in U.S. Pat. No. 4,557,925, which relevant part is incorporated herein by reference for this purpose. The porous membrane can also be coated with an enteric coating, as described herein, to inhibit release in the stomach.

For example, a pore forming modified release dosage form can comprise P-glycoprotein and/or non-P-glycoprotein substrate; a filler, such as starch, lactose, or microcrystalline cellulose (AVICEL™); a binder/modified release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulfate or polysorbates; and a glidant, such as colloidal silicon dioxide (AEROSIL™) or talc.

The polymeric material can also include one or more auxiliary agents such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 0.5% to about 300% by weight, and can range from about 0.5% to about 100%, based on the total dry weight of the polymer. In one embodiment, talc is the filler.

The coating membranes, and functional coatings as well, can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 0.5% to about 50%, for example, about 0.5, 1, 2, 5, 10, 20, 30, 40, or 50%, based on the weight of the dry polymer.

Anti-foaming agents can also be included. In one embodiment, the anti-foaming agent is simethicone. The amount of anti-foaming agent used typically comprises from about 0% to about 0.5% of the final formulation.

The amount of polymer to be used in the membrane modified formulations is typically adjusted to achieve the desired P-glycoprotein and/or non-P-glycoprotein substrate delivery properties, including the amount of drug to be delivered, the rate and location of P-glycoprotein and/or non-P-glycoprotein substrate delivery, the time delay of P-glycoprotein and/or non-P-glycoprotein substrate release, and the size of the multiparticulates in the formulation. The amount of polymer applied typically provides an about 0.5% to about 100% weight gain to the cores. In one embodiment, the weight gain from the polymeric material ranges from about 2% to about 70%.

The combination of all solid components of the polymeric material, including co-polymers, fillers, plasticizers, and optional excipients and processing aids, typically provides an about 0.5% to about 450% weight gain on the cores. In one embodiment, the weight gain is about 2% to about 160%.

The polymeric material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature.

A sealant or barrier can also be applied to the polymeric coating. A sealant or barrier layer can also be applied to the core prior to applying the polymeric material. A sealant or barrier layer is not intended to modify the release of P-glycoprotein and/or non-P-glycoprotein substrate. Suitable sealants or barriers are permeable or soluble agents such as hydroxypropyl methylcellulose, hydroxypropyl cellulose, hydroxypropyl ethylcellulose, and xanthan gum.

Other agents can be added to improve the processability of the sealant or barrier layer. Such agents include talc, colloidal silica, polyvinyl alcohol, titanium dioxide, micronized silica, fumed silica, glycerol monostearate, magnesium trisilicate and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

The invention also provides an oral dosage form containing a multiparticulate P-glycoprotein and/or non-P-glycoprotein substrate formulation as hereinabove defined, in the form of caplets, capsules, particles for suspension prior to dosing, sachets, or tablets. When the dosage form is in the form of tablets, the tablets can be disintegrating tablets, fast dissolving tablets, effervescent tablets, fast melt tablets, and/or mini-tablets. The dosage form can be of any shape suitable for oral administration of a P-glycoprotein and/or non-P-glycoprotein substrate, such as spheroidal, cube-shaped, oval, or ellipsoidal. The dosage forms can be prepared from the multiparticulates in any known manner and can include additional pharmaceutically acceptable excipients.

All of the particular embodiments described above, including but not limited to, matrix-based, osmotic pump-based, soft gelatin capsules, and/or membrane-modified forms, which can further take the form of monolithic and/or multi-unit dosage forms, can have a functional coating. Such coatings generally serve the purpose of delaying the release of the P-glycoprotein and/or non-P-glycoprotein substrate for a predetermined period. For example, such coatings can allow the dosage form to pass through the stomach without being subjected to stomach acid or digestive juices. Thus, such coatings can dissolve or erode upon reaching a desired point in the gastrointestinal tract, such as the upper intestine.

Such functional coatings can exhibit pH-dependent or pH-independent solubility profiles. Those with pH-independent profiles generally erode or dissolve away after a predetermined period, and the period is generally directly proportional to the thickness of the coating. Those with pH-dependent profiles, on the other hand, can maintain their integrity while in the acid pH of the stomach, but quickly erode or dissolve upon entering the more basic upper intestine.

Thus, a matrix-based, osmotic pump-based, or membrane-modified formulation can be further coated with a functional coating that delays the release of the P-glycoprotein and/or non-P-glycoprotein substrate. For example, a membrane-modified formulation can be coated with an enteric coating that delays the exposure of the membrane-modified formulation until the upper intestine is reached. Upon leaving the acidic stomach and entering the more basic intestine, the enteric coating dissolves. The membrane-modified formulation then is exposed to gastrointestinal fluid, and releases P-glycoprotein and/or non-P-glycoprotein substrate over an extended period, in accordance with the invention. Examples of functional coatings such as these are known in the art.

The thickness of the polymer in the formulations, the amounts and types of polymers, and the ratio of water-soluble polymers to water-insoluble polymers in the modified-release formulations are generally selected to achieve a desired release profile of P-glycoprotein and/or non-P-glycoprotein substrate. For example, by increasing the amount of water-insoluble-polymer relative to the water-soluble polymer, the release of the P-glycoprotein and/or non-P-glycoprotein substrate can be delayed or slowed.

The present inventive methods and formulations include pH-independent and/or pH-dependent modified-release formulations (e.g., non-enteric, pH-dependent systems) comprising P-glycoprotein and/or non-P-glycoprotein substrate, or a pharmaceutically acceptable salt thereof, that when measured by a U.S. Pharmacopoeia (USP) Type 1 Apparatus (baskets) or U.S. Pharmacopoeia (USP) Type 2 Apparatus (paddles) at 37° C. and 50 rpm or higher in phosphate buffer at pH 6.8 for the measuring period, release less than about 20%, or less than about 10% of the P-glycoprotein and/or non-P-glycoprotein substrate, in vitro in less than about 1 hours; release from about 5% to about 40%, or from about 15% to about 30%, in about 2 hours; release from about 10% to about 60%, or from about 20% to about 50%, in about 4 hours; release from about 20% to about 80%, or from about 30% to about 70%, in about 6 hours; release greater than about 50%, or greater than about 60%, in about 8 hours; and release greater than about 70%, or greater than about 80%, in about 12 hours. Note that formulations of this invention may fall within one or more of these dissolution windows.

Formulations, which can be pH-dependent modified-release formulations (e.g., enteric, pH-dependent systems), according to the present invention, when measured by a U.S. Pharmacopoeia (USP) Type 1 Apparatus (baskets) or U.S. Pharmacopoeia (USP) Type 2 Apparatus (paddles) at 37° C. and 50 rpm or higher in pH 1.2 (0.1 N HCl) for two hours, followed by phosphate buffer at pH 7.2 for the remaining measuring period, can exhibit dissolution profiles falling within one or more of these dissolution windows: 2 hours (in acid), about 0 to about 20%, or about 0 to about 10%, released; 1 hour (in buffer, after acid), greater than about 50%, or greater than about 60% released; 2 hours (in buffer, after acid), greater than about 70%, or greater than about 80%, released. In some embodiments, the inventive formulations can exhibit dissolution profiles falling within one or more of these dissolution windows: 2 hours (in acid), about 0 to about 20%, or about 0 to about 10%, released; 1 hour (in buffer, after acid), greater than about 20%, or greater than about 30%, released; 2 hours (in buffer, after acid), greater than about 30%, or greater than about 40%, released; 4 hours (in buffer, after acid), greater than about 50%, or greater than about 60% or greater released; and 6 hours (in buffer, after acid), greater than about 70%, or greater than about 80%, released. Again, it should be noted that formulations of this invention may fall within one or more of these dissolution windows.

The present invention overcomes the deficiencies and problems in the prior art by providing new and effective formulations and methods for reducing, preventing, and/or managing gastrointestinal conditions, and symptoms thereof. The methods for reducing, preventing, and/or managing gastrointestinal conditions involve administering an effective amount of a P-glycoprotein and/or non-P-glycoprotein substrate, or a pharmaceutically acceptable salt thereof, to a subject in need of such reduction, prevention, and/or management. The present invention can also be used to directly or indirectly reduce, prevent, and/or manage such gastrointestinal conditions by the use of these P-glycoprotein and/or non-P-glycoprotein substrates. Examples of gastrointestinal conditions that can be treated, prevented, and/or managed according to the present invention include, but are not limited to, food allergies, celiac disease, irritable bowel disease, mastocytosis, atopic dermatitis, inflammatory bowel disease, ulcerative colitis, granulomatous enteritis, Crohn's disease, infectious diseases of the small and large intestine, pyloric spasm, abdominal cramps, functional gastrointestinal disorders, mild dysenteries, diverticulitis, acute enterocolitis, neurogenic bowel disorders, including the splenic flexure syndrome and neurogenic colon, spastic colitis, cysts, polyps, and carcinoma, and/or symptoms of any of the foregoing. Those of ordinary skill in the art will be familiar with other types of gastrointestinal conditions that can benefit from the present invention.

As used herein, the term “pharmaceutically acceptable salt” includes salts that are physiologically tolerated by a subject. Such salts can be prepared from an inorganic and/or organic acid. Examples of suitable inorganic acids include, but are not limited to, hydrochloric, hydrobromic, hydroiodic, nitric, sulfuric, and phosphoric acid. Organic acids can be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

In accordance with the invention, the P-glycoprotein and/or non-P-glycoprotein substrate, or a pharmaceutically acceptable salt thereof, is formulated and/or dosed in a manner that maximizes its therapeutic effects, while minimizing at least one systemic side effect.

The P-glycoprotein and/or non-P-glycoprotein substrate, or a pharmaceutically acceptable salt thereof, can be administered with one or more of such pharmaceutically active compounds. Combinations can be administered such that P-glycoprotein and/or non-P-glycoprotein substrate, or a pharmaceutically acceptable salt thereof, and the at least one other pharmaceutically active compound are contained in the same dosage form. Alternatively, the combinations can be administered such that P-glycoprotein and/or non-P-glycoprotein substrate and the at least one additional pharmaceutically active compound are contained in separate dosage forms and are administered concomitantly or sequentially. Still further, a P-glycoprotein substrate can be delivered in the same dosage form as, or separate dosage form from, a non-P-glycoprotein substrate.

The pharmaceutically acceptable formulations described herein can be provided in the form of a pharmaceutical formulation for use according to the present invention. Such formulations optionally include one or more pharmaceutically acceptable excipients. Examples of suitable excipients are known to those of skill in the art and are described, for example, in the Handbook of Pharmaceutical Excipients (Kibbe (ed.), 3.sup.rd Edition (2000), American Pharmaceutical Association, Washington, D.C.), and Remington: The Science and Practice of Pharmacy (Gennaro (ed.), 20.sup.th edition (2000), Mack Publishing, Inc., Easton, Pa.) (hereinafter referred to as “Remington”), both of which, for their disclosures relating to excipients and dosage forms, are incorporated herein by reference. Suitable excipients include, but are not limited to, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, plasticizers, gelling agents, thickeners, hardeners, setting agents, suspending agents, surfactants, humectants, carriers, stabilizers, antioxidants, and combinations thereof.

Formulations suitable for oral administration include, but are not limited to, capsules, cachets, pills, tablets, lozenges (using a flavored base, usually sucrose and acacia or tragacanth), powders, granules, solutions, suspensions in an aqueous or non-aqueous liquid, oil-in-water or water-in-oil liquid emulsions, elixirs, syrups, pastilles (using an inert base, such as gelatin and glycerin, or sucrose and acacia), mouth washes, pastes, and the like, each containing a predetermined amount of P-glycoprotein and/or non-P-glycoprotein substrate, or a pharmaceutically acceptable salt thereof, to provide a therapeutic amount of the P-glycoprotein and/or non-P-glycoprotein substrate in one or more doses.

The P-glycoprotein and/or non-P-glycoprotein substrate, or a pharmaceutically acceptable salt thereof, can be mixed with pharmaceutically acceptable excipients in the preparation of dosage forms for oral administration (capsules, tablets, pills, powders, granules and the like). Suitable excipients include, but are not limited to, carriers, such as sodium citrate or dicalcium phosphate; fillers or extenders, such as starches, lactose, sucrose, glucose, mannitol, or silicic acid; binders, such as hydroxymethyl-cellulose, alginates, gelatin, polyvinylpyrrolidone, sucrose or acacia; humectants, such as glycerol; disintegrating agents, such as agar, calcium carbonate, potato or tapioca starch, alginic acid, certain silicates, or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, and sodium lauryl sulfate; coloring agents; buffering agents; dispersing agents; preservatives; and diluents.

The aforementioned excipients are given as examples only and are not meant to include all possible choices. Solid formulations can also be employed as fillers in soft and hard-filled gelatin capsules using excipients such as lactose or milk sugars, high molecular weight polyethylene glycols, and the like. Any of these dosage forms can optionally be scored or prepared with coatings and shells, such as enteric coatings and coatings for modifying the rate of release, examples of which are well known in the pharmaceutical-formulating art.

Such coatings can comprise sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, wax, or zein. In one embodiment, the coating material comprises hydroxypropyl methylcellulose. The coating material can further comprise anti-adhesives, such as talc; plasticizers (depending on the type of coating material selected), such as castor oil, diacetylated monoglycerides, dibutyl sebacate, diethyl phthalate, glycerin, polyethylene glycol, propylene glycol, triacetin, triethyl citrate; opacifiers, such as titanium dioxide; and/or coloring agents and/or pigments. The coating process can be carried out by any suitable means, for example, by using a perforated pan system such as the GLATT™, ACCELACOTA™, and/or HICOATER™ apparatuses.

Tablets can be formed by any suitable process, examples of which are known to those of ordinary skill in the art. For example, the ingredients can be dry-granulated or wet-granulated by mixing in a suitable apparatus before tabletting. Granules of the ingredients to be tabletted can also be prepared using suitable spray/fluidization or extrusion/spheronization techniques.

The tablets can be formulated with suitable excipients to act as a fast dissolving and/or fast melting tablet in the oral cavity. Also, the tablet can be in the form of a chewable or effervescent dosage form. With effervescent dosage forms, the tablet can be added to a suitable liquid that causes it to disintegrate, dissolve, and/or disperse.

Tablets can be designed to have an appropriate hardness and friability to facilitate manufacture on an industrial scale using equipment to produce tablets at high speed. Also, the tablets can be packed or filled in any kind of container. It should be noted that the hardness of tablets, amongst other properties, can be influenced by the shape of the tablets. Different shapes of tablets can be used according to the present invention. Tablets can be circular, oblate, oblong, or any other shape. The shape of the tablets can also influence the disintegration rate.

Any of the inventive formulations can be encapsulated in soft and hard gelatin capsules, which can also include any of the excipients described above. For example, the encapsulated dosage form can include fillers, such as lactose and microcrystalline; glidants, such as colloidal silicon dioxide and talc; lubricants, such as magnesium stearate; and disintegrating agents, such as starch (e.g., maize starch). Using capsule filling equipment, the ingredients to be encapsulated can be milled together, sieved, mixed, packed together, and then delivered into a capsule. Lubricants can be present in an amount of from about 0.5% (w/w) to about 2.0% (w/w).

The formulations of the invention, which comprise P-glycoprotein and/or non-P-glycoprotein substrate, or a pharmaceutically acceptable salt thereof, can also be formulated into a liquid dosage form for oral administration. Suitable formulations can include emulsions, microemulsions, solutions, suspensions, syrups, and elixirs. The P-glycoprotein and/or non-P-glycoprotein substrate can be formulated as an ion-exchange resin complex, a microencapsulated particle, a liposome particle, or a polymer coated particle or granule. These formulations optionally include diluents commonly used in the art, such as, for example, water or other solvents, solubilizing agents and emulsifiers. Emulsifiers include, but are not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofurfuryl alcohol, polyethylene glycols, fatty acid esters of sorbitan, and mixtures thereof. In addition, the inventive formulations can include adjuvants such as wetting agents, emulsifying and suspending agents, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. The liquid formulations can be delivered as-is, or can be provided in hard or soft capsules, for example.

The amount of suspending agent present will vary according to the particular suspending agent used, and the presence or absence of other ingredients that have an ability to act as a suspending agent or contribute significantly to the viscosity of the formulation. The suspension can also contain ingredients that improve its taste, for example sweeteners; bitter-taste maskers, such as sodium chloride; taste-masking flavors, such as contramarum; flavor enhancers, such as monosodium glutamate; and flavoring agents. Examples of sweeteners include bulk sweeteners, such as sucrose, hydrogenated glucose syrup, the sugar alcohols sorbitol and xylitol; and sweetening agents such as sodium cyclamate, sodium saccharin, aspartame, and ammonium glycyrrhizinate. The liquid formulations can further comprise one or more buffering agents, as needed, to maintain a desired pH.

The liquid formulations of the present invention can also be filled into soft gelatin capsules. The liquid can include a solution, suspension, emulsion, microemulsion, precipitate, or any other desired liquid media carrying the pharmaceutically active compound. The liquid can be designed to improve the solubility of the pharmaceutically active compound upon release, or can be designed to form a drug-containing emulsion or dispersed phase upon release. Examples of such techniques are well known in the art. Soft gelatin capsules can be coated, as desired, with a functional coating. Such functional coatings generally serve the purpose of delaying the release of the P-glycoprotein and/or non-P-glycoprotein substrate for a predetermined period. For example, such coatings can allow the dosage form to pass through the stomach without being subjected to stomach acid or digestive juices. Thus, such coatings can dissolve or erode upon reaching a desired point in the gastrointestinal tract, such as the upper intestine.

For rectal administration, the inventive formulations can be provided as a suppository. Suppositories can comprise one or more non-irritating excipients such as, for example, polyethylene glycol, a suppository wax, or a salicylate. Such excipients can be selected on the basis of desirable physical properties. For example, a compound that is solid at room temperature but liquid at body temperature will melt in the rectum and release the active compound. The formulation can alternatively be provided as an enema for rectal delivery.

The amount of the dose administered, as well as the dose frequency, will vary depending on the particular dosage form used and the route of administration. The amount and frequency of administration will also vary according to the age, body weight, and response of the individual subject. Typical dosing regimens can readily be determined by a competent physician without undue experimentation. It is also noted that the clinician or treating physician will know how and when to interrupt, adjust, or terminate therapy in conjunction with individual subject response.

In general, the total daily dosage for reducing, preventing, and/or managing the gastrointestinal symptoms and/or the intestinal conditions that cause the same, with any of the formulations according to the present invention, is from about 1 mg to about 8000 mg, or from about 5 mg to about 7000 mg, or from about 10 mg to about 6000 mg, or from about 20 mg to about 4000 mg. A single oral dose can be formulated to contain about 1, 2, 5, 10, 20, 50, 100 mg, 250 mg, 500 mg, 750 mg, 1000 mg, 1500 mg, 2000 mg, or 3000 mg, or any amount in between. For fexofenadine, the daily doses will generally range from about 30 mg up to about 480 mg per day, or more preferably from about 60 mg to about 240 mg/day. For sodium cromoglycate, the daily doses will generally range from about 200 mg/day to about 2,000 mg per day.

The pharmaceutical formulations containing P-glycoprotein and/or non-P-glycoprotein substrate, or a pharmaceutically acceptable salt thereof, can be administered in single or divided doses, 1, 2, 3, 4, 5, or more times each day. Alternatively, the dose can be delivered one or more times every 2, 3, 4, 5, 6, 7, or more days. In one embodiment, the pharmaceutical formulations are administered once per day.

Although the present invention has been described in considerable detail with regard to certain versions thereof, other versions are possible, and alterations, permutations, and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

What is claimed is:

1. A method of treating a condition associated with gastrointestinal symptoms comprising administering to a patient in need of such treatment an effective amount of a P-glycoprotein substrate, wherein the P-glycoprotein substrate is a compound exhibiting an efflux inhibition ratio (EIR) of greater than or equal to 0.4, wherein the P-glycoprotein substrate is administered in a manner to minimize systemic bioavailability.

2. The method according to claim 1, wherein the P-glycoprotein substrate is a compound exhibiting an EIR of greater than or equal to 0.5.

3. The method according to claim 2, wherein the P-glycoprotein substrate is a compound exhibiting an EIR of greater than or equal to 0.6.

4. The method according to claim 1, wherein the P-glycoprotein substrate is administered in a manner to result in less than or equal to about 80% of the bioavailability of an orally administered rapid release dosage form of the P-glycoprotein substrate.

5. The method according to claim 4, wherein the P-glycoprotein substrate is administered in a manner to result in less than or equal to about 70% of the bioavailability of an orally administered rapid release dosage form of the P-glycoprotein substrate.

6. The method according to claim 5, wherein the P-glycoprotein substrate is administered in a manner to result in less than or equal to about 60% of the bioavailability of an orally administered rapid release dosage form of the P-glycoprotein substrate.

7. The method according to claim 4, wherein the P-glycoprotein substrate is administered in a pharmaceutical formulation that releases less than or equal to about 20% of the P-glycoprotein substrate in up to 2 hours of testing in pH 1.2 in a USP Type 2 dissolution testing apparatus.

8. The method according to claim 7, wherein the P-glycoprotein substrate is administered in a pharmaceutical formulation that releases greater than about 70% of the P-glycoprotein substrate in 2 hours of testing in pH 1.2 followed by pH 7.2 for 2 hours, in a USP Type 2 dissolution testing apparatus.

9. The method according to claim 4, wherein the P-glycoprotein substrate is chosen from histamine H1 antagonists, corticosteroids, glucocorticosteroids, aminosalicylates, mast cell stabilisers, leukotriene antagonists, and combinations thereof.

10. The method according to claim 9, wherein the P-glycoprotein substrate is chosen from histamine H1 antagonists.

11. The method according to claim 10, wherein the P-glycoprotein substrate is fexofenadine.

12. The method according to claim 11, wherein the P-glycoprotein substrate is enriched (S)-fexofenadine.

13. The method according to claim 12, wherein the P-glycoprotein substrate is substantially pure (S)-fexofenadine.

14. The method according to claim 1, further comprising administering at least one non-P-glycoprotein substrate, wherein the non-P-glycoprotein substrate is a compound exhibiting an EIR of less than 0.4.

15. The method according to claim 14, wherein the non-P-glycoprotein substrate is chosen from histamine H1 antagonists, corticosteroids, glucocorticosteroids, aminosalicylates, mast cell stabilisers, and leukotriene antagonists.

16. A method of treating a gastrointestinal condition comprising orally administering to a patient in need of such treatment a pharmaceutical formulation comprising fexofenadine, which releases from about 0 to about 20% of the fexofenadine in the formulation within two hours of administration, wherein the bioavailability of the fexofenadine in the formulation is less than or equal to about 80% of the bioavailability of an orally administered rapid release dosage form of fexofenadine.

17. The method according to claim 16, wherein the pharmaceutical formulation releases from about 0 to about 10% of the fexofenadine in the formulation within two hours of administration.

18. The method according to claim 17, wherein the bioavailability of the fexofenadine in the formulation is less than or equal to about 70% of the bioavailability of an orally administered rapid release dosage form of fexofenadine.

19. The method according to claim 18, wherein the bioavailability of the fexofenadine in the formulation is less than or equal to about 60% of the bioavailability of an orally administered rapid release dosage form of fexofenadine.

20. The method according to claim 19, wherein the bioavailability of the fexofenadine in the formulation is less than or equal to about 50% of the bioavailability of an orally administered rapid release dosage form of fexofenadine.

21. The method according to claim 18, wherein the pharmaceutical formulation further comprises at least one non-P-glycoprotein substrate, wherein the non-P-glycoprotein substrate is a compound exhibiting an EIR of less than 0.4.

22. The method according to claim 21, wherein the at least one compound is chosen from anti-H1 antagonists, corticosteroids, glucocorticosteroids, aminosalicylates, mast cell stabilisers, and leukotriene antagonists.

23. An oral drug delivery system consisting of:
a P-glycoprotein substrate exhibiting an EIR of greater than 0.4; and
pharmaceutical excipients that result in release of less than or equal to about 20% of the P-glycoprotein substrate in up to 2 hours of testing in pH 1.2 in a USP Type 2 dissolution testing apparatus.

24. The oral drug delivery system according to claim 23, wherein the P-glycoprotein substrate is fexofenadine.

25. An oral drug delivery system consisting of:
a P-glycoprotein substrate exhibiting an EIR of greater than 0.4 and a non-p-glycoprotein substrate exhibiting an EIR of less than 0.4; and
pharmaceutical excipients that result in release of less than or equal to about 20% of the P-glycoprotein substrate in up to 2 hours of testing in pH 1.2 in a USP Type 2 dissolution testing apparatus.

26. The oral drug delivery system according to claim 25, wherein the P-glycoprotein substrate is fexofenadine and the non-P-glycoprotein substrate is sodium cromoglycate.
